(19) United States
(12) Patent Application Publication
(10) Pub. No.: US 2026/0345019 A1
(43) Pub. Date: Nov. 5, 2026

(54) REUSABLE NON-INVASIVE BLOOD PRESSURE MONITORING SYSTEM

(71) Applicant: Becton, Dickinson and Company, Franklin Lakes, NJ (US)

(72) Inventors: Huy Thanh Vu, Westminster, CA (US); Greg Joseph Dugan, Corona, CA (US); Ilja Guelen, Ravenstein (NL); Abdulkader Sudam, Dana Point, CA (US); Julia Leigh Ross, Huntington Beach, CA (US)

(21) Appl. No.: 19/712,971

(22) Filed: Jun. 18, 2026

Related U.S. Application Data
(63) Continuation of application No. PCT/US2024/061023, filed on Dec. 19, 2024.
(60) Provisional application No. 63/612,960, filed on Dec. 20, 2023.

(51) Int. Cl. A61B 5/022; A61B 5/00

(52) U.S. Cl. CPC A61B 5/02241; A61B 5/6843

(57) ABSTRACT

Disclosed herein is a blood pressure monitor component for monitoring a patient's blood pressure including: a cuff connected to a rack which includes a series of apertures along at least one end of the cuff, wherein the cuff includes a bladder; a pinion capable of engaging with the series of apertures of the rack; and a force sensor configured to sense tension applied by the cuff. The blood pressure monitor component may include a light source and a light sensor which are integrated within the cuff. The light source may be configured to output light which is received by the light sensor. The amount of light which is received by the light sensor corresponds to the blood pressure.

### CROSS-REFERENCE TO RELATED APPLICATION(S)

This application is a continuation of International Application No. PCT/US2024/061023, filed Dec. 19, 2024, which claims priority to U.S. Provisional Application 63/612,960 filed on Dec. 20, 2023, the disclosures of which are incorporated by reference in their entirety.

### FIELD OF THE DISCLOSURE

The present disclosed technology generally relates to a reusable non-invasive blood pressure monitoring system including a resizable cuff surrounding an inflatable bladder.

### BACKGROUND

Continuous noninvasive blood pressure monitors enable real-time measurement of blood pressure waves and derived hemodynamic parameters. Multiple techniques can be utilized including the volume clamp method.

Volume clamp method measures arterial blood pressure at an extremity (e.g., finger) utilizing an inflatable bladder, a light source (e.g., light emitting diode (LED)), and light sensor. The pressure in the bladder is adjusted to keep the diameter of the artery constant (the unloaded state), in which the diameter is determined via the light source and light sensor. The pressure within the inflatable bladder represents the arterial pressure of the finger artery. A pressure pump supplies the pressure to the inflatable bladder. The inflatable bladder may be within a cuff. The cuff may be adjustable to accommodate patients with different sized fingers.

### SUMMARY OF THE DISCLOSURE

In some aspects, the techniques described herein relate to a blood pressure monitor component for monitoring a patient's blood pressure including: a cuff defining a cavity for receiving an appendage; a bladder at least partially surrounding the cavity; a fixed end of the cuff connected with a base and a free end of the cuff movable with respect to the fixed end to adjust a diameter of the cavity; and a force sensor configured to sense tension applied by the cuff.

In some aspects, the techniques described herein relate to a blood pressure monitor component, further including a computer including: a rack which includes a series of apertures along the free end of the cuff; a pinion capable of engaging with the series of apertures of the rack; a processor; and memory with instructions configured to operate the processor to: receive force data from the force sensor; determine an amount of tension being applied by the cuff by the force data; and operate the pinion based on the amount of tension being applied by the cuff.

In some aspects, the techniques described herein relate to a blood pressure monitor component, further including a light source and a light sensor which are integrated within the cuff, wherein the light source is configured to output light which is received by the light sensor, wherein the amount of light which is received by the light sensor corresponds to the blood pressure.

In some aspects, the techniques described herein relate to a blood pressure monitor component, wherein the instructions are further configured to operate the processor to perform a blood pressure measurement after operating the pinion.

In some aspects, the techniques described herein relate to a blood pressure monitor component, further including a motor attached to a gear by a shaft, wherein the gear drives the pinion.

In some aspects, the techniques described herein relate to a blood pressure monitor component, wherein operating the pinion based on the amount of tension being applied by the cuff includes upon a determination that the amount of tension being applied by the cuff is too high, operating the motor which drives the pinion to adjust the cuff to be loosened.

In some aspects, the techniques described herein relate to a blood pressure monitor component, wherein operating the pinion based on the amount of tension being applied by the cuff includes upon a determination that the amount of tension being applied by the cuff is too low, operating the motor which drives the pinion to adjust the cuff to be tightened.

In some aspects, the techniques described herein relate to a blood pressure monitor component, wherein operating the pinion based on the amount of tension being applied by the cuff includes upon a determination that the amount of tension being applied by the cuff is appropriate, operating the motor which drives the pinion to maintain the tension.

In some aspects, the techniques described herein relate to a blood pressure monitor component, wherein operating the motor which drives the pinion to maintain the tension includes providing no signal to the motor.

In some aspects, the techniques described herein relate to a blood pressure monitor component, wherein the pinion includes teeth which corresponds to the series of apertures of the rack to move the rack and decrease or increase the size of the cuff.

In some aspects, the techniques described herein relate to a blood pressure monitor component, wherein the gear includes teeth which interlock with the teeth of the pinion.

In some aspects, the techniques described herein relate to a blood pressure monitor component, wherein the pinion is capable of rotation, wherein rotation of the pinion results in linear movement of the cuff such that cuff is tightened or loosened.

In some aspects, the techniques described herein relate to a blood pressure monitor system for monitoring a patient's blood pressure, including: a blood pressure monitor component including: a cuff connected to a rack which includes a series of apertures along at least one end of the cuff, wherein the cuff includes a bladder; a pinion capable of engaging with the series of apertures of the rack; and a force sensor configured to sense tension applied by the cuff; and a pump system in operable connection with the bladder configured to provide pressure to the bladder.

In some aspects, the techniques described herein relate to a blood pressure monitor system, further including a computer including: a processor; and memory with instructions configured to operate the processor to: receive force data from the force sensor; determine an amount of tension being applied by the cuff by the force data; and operate the pinion based on the amount of tension being applied by the cuff.

In some aspects, the techniques described herein relate to a blood pressure monitor system, further including a light source and a light sensor which are integrated within the cuff, wherein the light source is configured to output light which is received by the light sensor, wherein the amount of light which is received by the light sensor corresponds to the blood pressure.

In some aspects, the techniques described herein relate to a blood pressure monitor system, wherein the instructions are further configured to operate the processor to perform a blood pressure measurement after operating the pinion.

In some aspects, the techniques described herein relate to a blood pressure monitor system, further including a motor attached to a gear by a shaft, wherein the gear drives the pinion.

In some aspects, the techniques described herein relate to a blood pressure monitor system, wherein operating the pinion based on the amount of tension being applied by the cuff includes upon a determination that the amount of tension being applied by the cuff is too high, operating the motor which drives the pinion to adjust the cuff to be loosened.

In some aspects, the techniques described herein relate to a blood pressure monitor system, wherein operating the pinion based on the amount of tension being applied by the cuff includes upon a determination that the amount of tension being applied by the cuff is too low, operating the motor which drives the pinion to adjust the cuff to be tightened.

In some aspects, the techniques described herein relate to a blood pressure monitor system, wherein the instructions are further configured to operate the processor to operate the pump system to provide an amount of pressure to the bladder.

### BRIEF DESCRIPTION OF THE DRAWINGS

The description will be more fully understood with reference to the following figures and data graphs, which are presented as various embodiment of the disclosure and should not be construed as a complete recitation of the scope of the disclosure, wherein:

FIG. 1A is a perspective view of a finger cuff system.

FIG. 1B is an exploded view of a finger cuff system.

FIG. 2A is a perspective view of a blood pressure monitor component.

FIG. 2B is an exploded view of a blood pressure monitor component.

FIG. 3A illustrates an example of a blood pressure monitor component being utilized on a patient's hand.

FIG. 3B illustrates an example of a blood pressure monitor component while being utilized on a patient's hand and integrated with a computational system.

FIG. 4A is a front view of a portable monitoring system.

FIG. 4B is a top down view of a portable monitoring system.

FIG. 4C is a side view of a portable monitoring system.

FIG. 5 illustrates an example data flow diagram for a blood pressure monitor component.

FIG. 6 illustrates an example block diagram of the computer which controls the motor based on the data from the force sensor.

FIG. 7 provides a conceptual illustration of a hemodynamic monitoring system.

### DETAILED DESCRIPTION

A universal-reusable blood pressure monitoring cuff may provide continuous and accurate blood pressure (beat-to-beat) measurement for any given size of patient finger without the potential use error of over or under tightening the cuff or rotating the cuff relative to the finger. Proper fit and application of the cuff on the patient's finger is important to the accuracy to measure hemodynamic parameters such as blood pressure, stroke volume, cardiac output, and stroke volume variation. Disclosed herein are systems and devices that provide a universal fit mechanism for a blood pressure cuff to provide robust securement, which can be utilized on a variety of finger sizes. The disclosed technology eliminates the potential of over or under tightening by clinicians when the cuff is applied to patient by automatically adjusting the fit of the cuff. Additionally, the disclosed technology ensures proper place of a plethysmograph utilized to measure arterial diameter.

Blood pressure monitoring systems can include a finger cuff that interfaces with the patient. The finger cuff can comprise a plethysmograph comprising a light source (e.g. LED) and a light sensor (e.g. photodiode) pair which sit on either side of the patient's finger. The cuff can further comprise an inflatable bladder that wraps around the finger. Accurate results of blood pressure readouts depend on the correct application of the finger cuff to the patient's finger. In some implementations, the cuff may be oriented so that the LED and photodiode are on opposite sides of the finger, not obstructed by bone, and may be sufficiently tight to provide the appropriate amount of pressure. The finger cuff may wrap snuggly around the finger to ensure the cuff is securely in place. However, it has been discovered that there is a potential use error to over tighten, under tighten, or rotate the cuff. Over or under tightening the cuff leads to increased or decreased pressure measurements on the cuff, which results in lower or higher blood pressure values respectively. Rotating the cuff so that the bone interferes with measurement can interfere with the pulse plethysmogram signal, causing inaccurate readings.

Various blood pressure systems monitor beat-to-beat blood pressure using a volume clamp method and assess cardiac output by pulse contour analysis. The pulse plethysmogram is measured by the plethysmograph comprising an LED and photodiode pair on either side of the finger, as pressure is applied to the finger via an inflatable bladder.

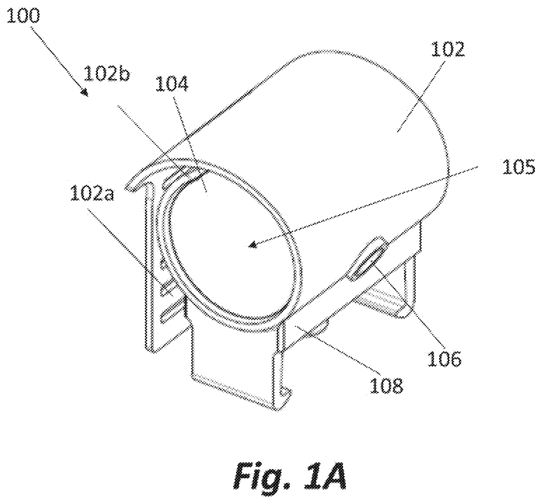

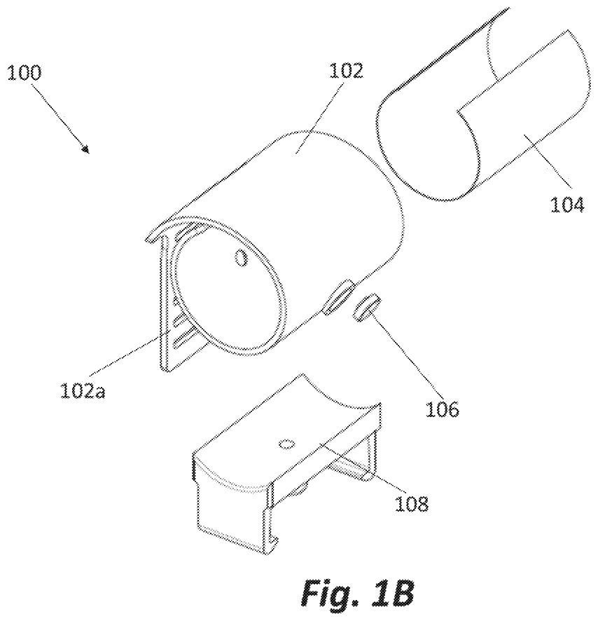

Turning to the drawings FIGS. 1A and 1B illustrate an example of a finger cuff system 100. The finger cuff system 100 includes a cuff 102. The cuff 102 can define around a cavity 105 for receiving an appendage of a patient (e.g., a finger). The cavity 105 can be configured to have an adjustable cross-sectional diameter, as described further below. The cuff 102 can include a movable end. The movable end can be a rack 102a. The rack 102a is capable of linear motion that can in order to tighten and/or loosen the cuff 102. As depicted, the rack 102a is a long strip with a series of apertures that engage with a pinion, but any linear actuator for providing linear movement of a cuff such that it can be tightened or loosened can be utilized. The linear position of an aperture may correspond to a certain size of the cuff 102. As illustrated below, a pinion may be utilized to move the rack 102a in order to tighten and/or loosen the cuff 102. The finger cuff system 100 may include a bladder 104 which may be inflated to a provided a pressure onto the patient's finger through the use of a pump. The bladder 104 can be semi-cylindrical (having an open slit as illustrated) or fully cylindrical having openings on first and second axial ends for receiving a finger or other appendage. The cuff 102 can further include coiled body structure forming the cavity 105. The cuff 102 can include an inner end 102b that is wrapped over by the movable end (e.g., rack 102a). The inner end 102b can be slidable engaged with an inner surface of the cuff 102 to allow for a cross-section diameter of the cavity 105 to be adjusted. The finger cuff system 100 may be mounted on a base 108. The cuff 102 can be attached with the base 108. The attachment point of the cuff 102 can be between the ends 102a, 102b. The base 108 can be considered a fixed end of the cuff 102 opposite the movable end (with the rack 102a). The finger cuff system 100 may include a light source 106 to provide light to measure an arterial diameter of the patient's finger. The light may be sensed by a detector opposing the light source 106. A blood pressure measurement may be obtained based on the amount of pressure within the bladder to maintain the arterial diameter constant.

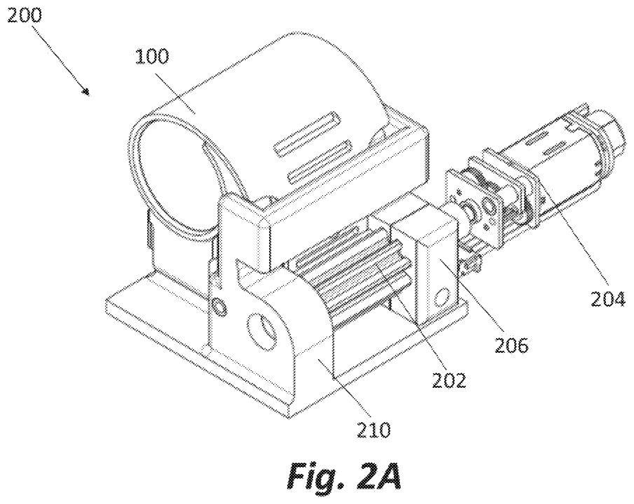

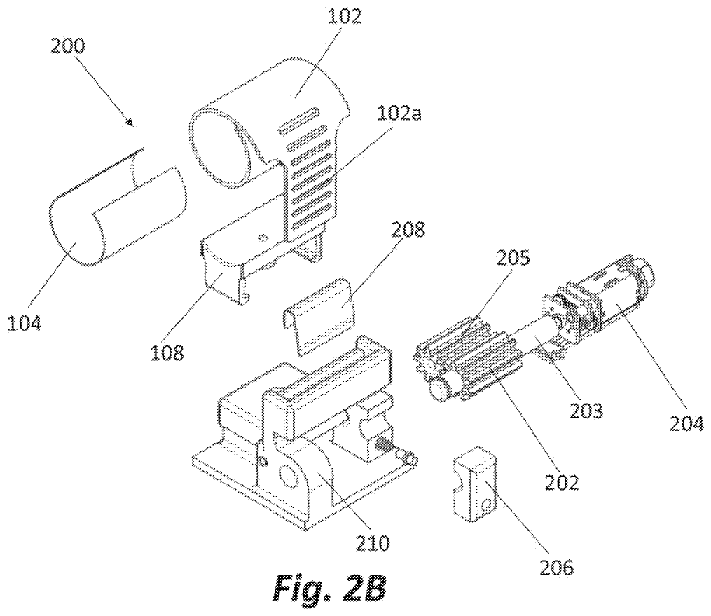

FIGS. 2A and 2B illustrate an example of a blood pressure monitor component 200 including the finger cuff system 100 described in connection with FIGS. 1A and 1B. The blood pressure monitor component 200 includes a gear 202 which may be a cylindrical member with teeth. The teeth engage with the apertures of the rack 102a to tighten or loosen the cuff 102. The gear 202 is attached to a motor 204 through a shaft 203. The shaft is held in place to a tension base 210 through a positioner 206.

The blood pressure monitor component 200 includes a force sensor 208 which may be used to sense the tension that the cuff 102 is applying to the patient's finger. A computer system is utilized to take measurements from the force sensor 208 and control the motor 204 based on the amount of tension applied to the patient's finger. For example, the motor 204 may rachet the gear 202 in one direction to loosen the cuff 102 if the tension is too high or the motor 204 may rachet the gear 202 in an opposite direction to tighten the cuff 102 is the tension is too low. The force sensor 208 may be a flexiforce pressure sensor. The force sensor 208 may be a piezoresistive force sensor. The force sensor 208 may be a pressure sensor which may have a certain resistance depth determined by the amount of the force that is placed on it. Control of the tension can be based on the pulse plethysmogram signal. The signal can be received from the LED and photodiode pair on either side of the finger. Control of the tension can be based on the pulse plethysmogram signal falling within a desired range. If the plethysmogram signal falls below a lower threshold, then the tension can be relaxed until the plethysmogram signal is above the lower threshold. If the plethysmogram signal is above an upper threshold, then the tension can be increased until the plethysmogram signal is below the upper threshold. Alternatively, control of the tension can be based on the data from the force sensor 208 falling within a desired range. If the data from the force sensor 208 falls below a lower threshold, then the tension can be relaxed until the force sensor data is above the lower threshold. If the force sensor data is above an upper threshold, then the tension can be increased until the force sensor data is below the upper threshold.

In some implementations, the gear 202 may be coupled to a pinion 205 which may be utilized to engage with the apertures of the rack 102a instead of the gear 202. The gear 202 may be utilized to drive the pinion 205. The gear 202 includes teeth which interlock with teeth of the pinion 205. Thus, the gear 202 may be utilized to indirectly rachet the rack 102a instead of directly.

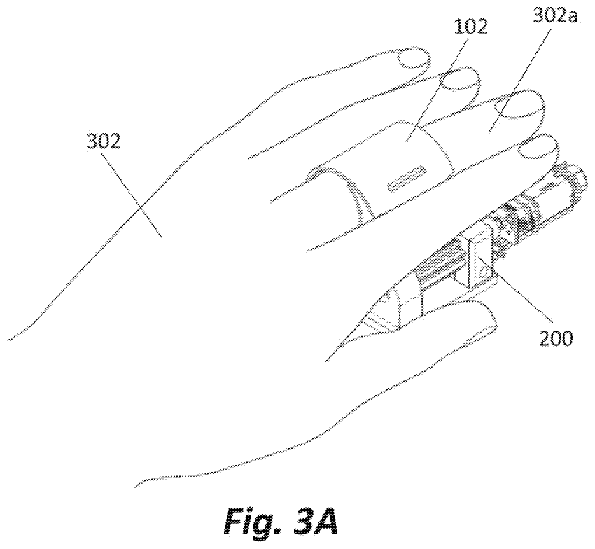

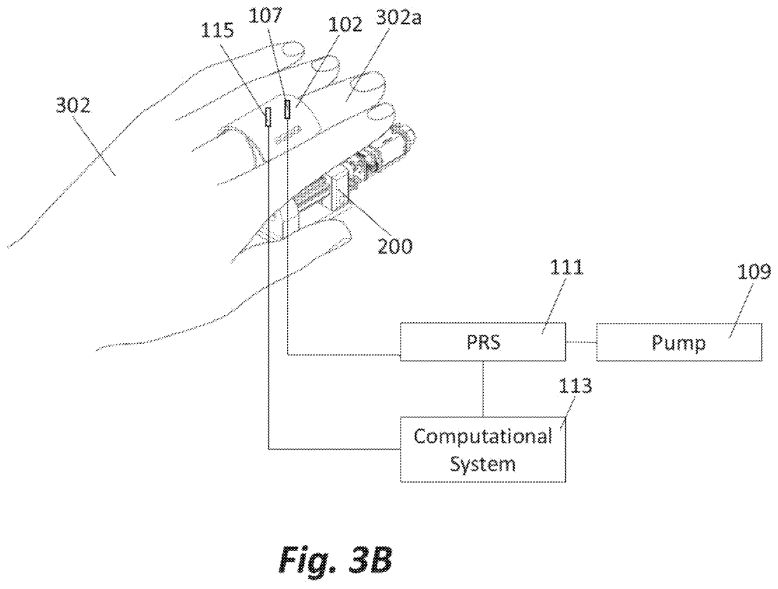

FIG. 3A illustrates an example of the blood pressure monitor component 200 described in connection with FIGS. 2A and 2B while being utilized on a patient's hand 302. As illustrated, the patient's finger 302a is positioned within the cuff 102. While the patient's middle finger is illustrated as being positioned within the cuff 102, the cuff 102 may be utilized on any digit. For example, the patient's index finger, thumb, pointer finger, or pinky finger may be utilized. Further, the blood pressure monitor component 200 may be utilized on any appendage to measure blood pressure. For example, the blood pressure monitor component 200 may be utilized on a toe, an arm, a leg, etc.

FIG. 3B illustrates an example of the blood pressure monitor component 200 described in connection with FIGS. 2A and 2B while being utilized on a patient's hand 302 and integrated with a computational system. The bladder 104 incorporates a pressure port 107 in fluidic connection with a pump (shown below) to provide pressure to the bladder 104. The pressure port 107 may be a nozzle, valve, or gasket. A pressure regulator system (PRS) 111 can be provided in between the cuff 102 and pump 109, which can sense and/or regulate the amount of pressure being provided to the inflation chamber. Pressure regulator system 111 can be controlled by a computational system 113, which can provide instructions to the pressure regulator system 111 to control the pressure in accordance with a computational application for measuring and monitoring blood pressure.

An electrical connection 115 can transmit power and/or data to and from the cuff 102. The electrical connection 115 can provide power and data for a light emitter and a light sensor, which are provided within a housing. The light emitter and light sensor can work in concert to measure the diameter of the artery within the thumb such that an accurate arterial blood pressure reading can be determined. The data collected by the light emitter and light sensor can be provided to the computational system 113 via electrical connection 115, which in turn can work in concert with pressure regulator system 111 to provide pressure for controlling the arterial diameter.

Computational system 113 can incorporate various computational programs for computing various arterial pressures (e.g., aortic pressure) from the measured arterial pressure within the thumb. Computational system 113 can further include a screen and user interface to display the various measured and computed arterial pressures and allow a user to interact with the blood pressure system.

While a specific blood monitoring system configuration is described above with reference to FIG. 3B, it should be readily appreciated that various blood monitoring systems and/or other medical monitoring utilized in the provision of blood pressure monitoring can be implemented in any of a variety of configurations. Accordingly, the various blood pressure cuffs described herein should be understood as not to be limited to any specific blood monitoring system, but instead can be implemented using any variety of blood monitoring or medical monitoring systems capable measuring arterial blood pressure.

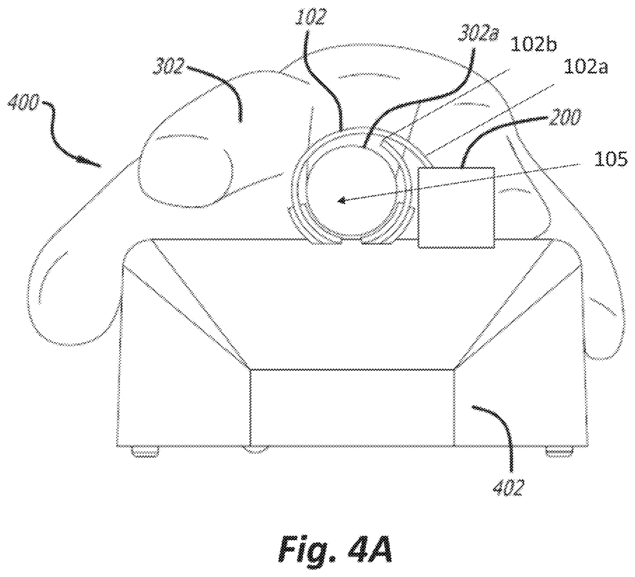

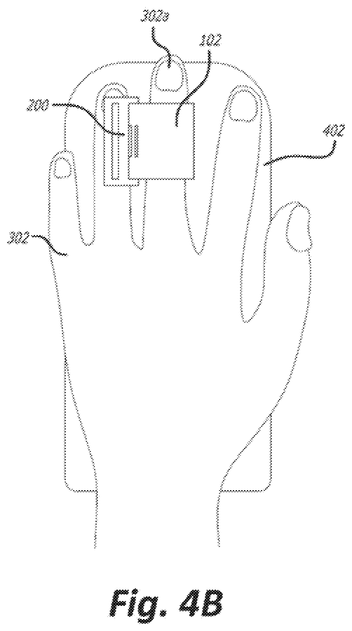

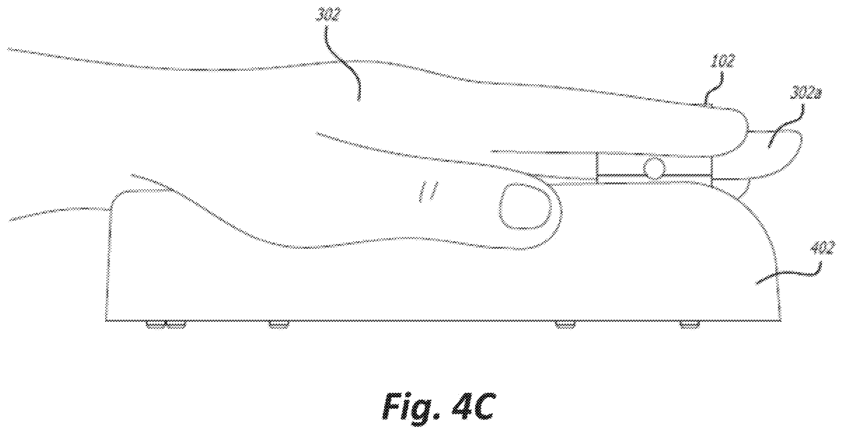

FIGS. 4A-4C illustrate a portable blood pressure monitoring system 400 including the blood pressure monitor component 200 described in connection with FIGS. 2A and 2B while being utilized on a patient's hand 302. The portable blood pressure monitoring system 400 includes a pump system 402 which may provide pressure to the bladder 104. The pump system 402 may be portable such that it may be connect to the blood pressure monitor component 200 and carried around with the blood pressure monitor component 200.

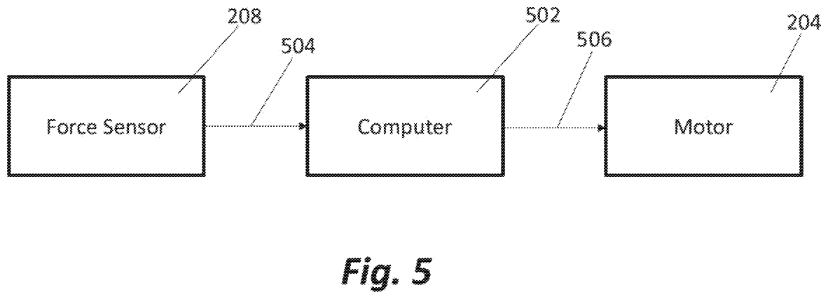

FIG. 5 illustrates an example data flow diagram for the blood pressure monitor component 200 described in connection with FIGS. 2A and 2B. The motor may be initially computer controlled for initial tightening on the patient's finger. The force sensor 208 outputs force data 504 to a computer 502. The force data 504 is analyzed and then a control signal 506 is sent to the motor 204 depending on the analysis. For example, if the force data 504 demonstrates that the tension on the patient's finger is too low, then the control signal 506 controls the motor 204 to increase tension on the cuff 102. If the force data 504 demonstrates that the tension on the patient's finger is too high, then the control signal 506 controls the motor 204 to decrease tension on the cuff 102. If the force data 504 demonstrates that the tension on the patient's finger is just right, then the control signal 506 controls the motor 204 maintain the tension on the cuff 102. In some examples, when the tension on the patient's finger is determined to be just right then there may be no control signal 506 sent to the motor 204.

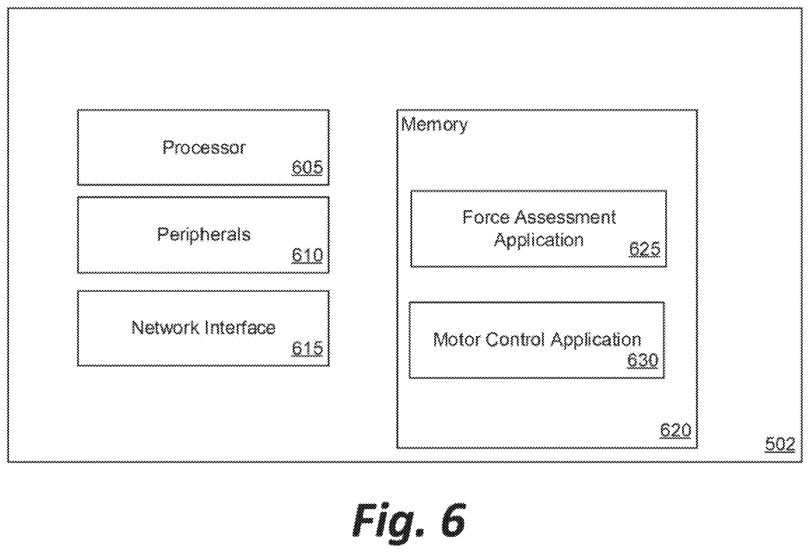

FIG. 6 illustrates an example block diagram of the computer 502 which controls the motor 204 based on the data from the force sensor 208. Computer 502 includes processor 605, peripherals 610, network interface 615, and memory 620. One skilled in the art will recognize that the computer 502 may exclude certain components and/or include other components that are omitted for brevity without departing from this invention.

The processor 605 can include (but is not limited to) a processor, microprocessor, controller, or a combination of processors, microprocessor, and/or controllers that performs instructions stored in the memory 620 to manipulate data stored in the memory. Processor instructions can configure the processor 605 to perform processes.

Peripherals 610 can include any of a variety of components for capturing data, such as (but not limited to) displays, and/or sensors. In a variety of implementations, peripherals can be used to gather inputs and/or provide outputs. The computer 502 can utilize a network interface 615 to transmit and receive data over a network based upon the instructions performed by processor 605. Peripherals and/or network interfaces can be used to gather inputs such as the force data from the force sensor 208 to control the motor 204.

Memory 620 includes a force assessment application 625 and a motor control application 630. The force assessment application 625 and the motor control application 630 can be used to utilize the force data from the force sensor 208 to control the motor 204.

The force assessment application 625 and motor control application 630 may be used as described above. For example, the force assessment application 625 may receive the force data from the force sensor 208. The force assessment application 625 may determine whether the force data indicates that the tension on the patient's finger is too high, too low, or just right.

The motor control application 630 may receive the determination of the force data's indication from the force assessment application 625. For example, if the force data 504 demonstrates that the tension on the patient's finger is too low, then the motor control application 630 may send a control signal 506 that controls the motor 204 to increase tension on the cuff 102. If the force data 504 demonstrates that the tension on the patient's finger is too high, then the motor control application 630 may send a control signal 506 that controls the motor 204 to decrease tension on the cuff 102. If the force data 504 demonstrates that the tension on the patient's finger is just right, then the motor control application 630 may send a control signal 506 that controls the motor 204 to maintain the tension on the cuff 102. In some examples, when the tension on the patient's finger is determined to be just right then the motor control application 630 may send no control signal 506 to the motor 204.

Although a specific example of the computer 502 is illustrated in this figure, any of a variety of the computer can be utilized to control the motor based on the force data from the force sensor 208 as appropriate to the requirements of specific applications.

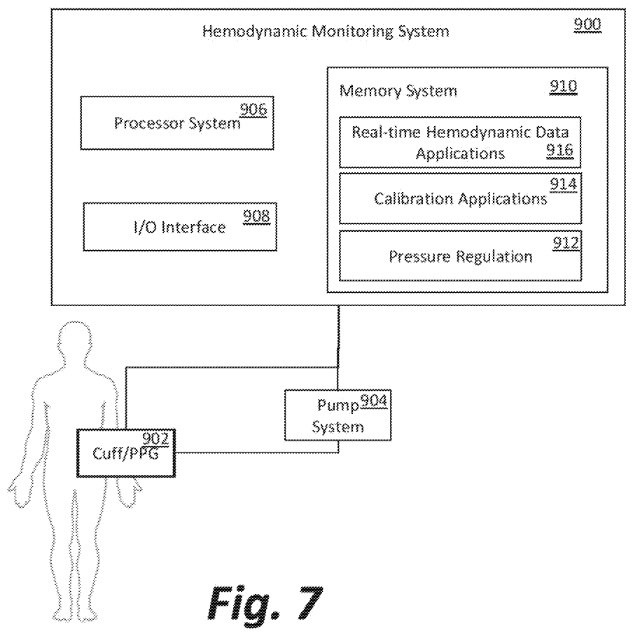

The systems and methods of the current disclosure can be utilized within a noninvasive hemodynamic monitoring system. Generally, the noninvasive hemodynamic monitoring system includes a pressurized cuff, a photoplethysmograph, a pressure regulator, and a computational system. Provided in FIG. 7 is an example of a noninvasive hemodyamic monitoring system 900 as would be utilized on a digit of an individual. On the digit is a blood pressure cuff with photoplethysmogram (PPG) 902 that keeps the artery within the digit at a constant diameter when performing arterial pressure measurements. The pressure in the cuff represents the arterial pressure within the digit. The PPG can further provide physiological data.

The blood pressure cuff with PPG can be in connection with hemodynamic monitoring system 900 and pump system 904. Data can be transmitted between the PPG 902 and hemodynamic monitoring system. Pump system 904 provides pressure to the blood pressure cuff. Pump system 904 can further be in connection with the hemodynamic monitoring system 900 such that the monitoring system can instruct the pump system to provide the requisite amount of pressure to cuff/PPG 902 to keep the artery diameter constant.

Hemodynamic monitoring system 900 can comprise a computational system. Hemodynamic monitoring system 900 can comprise a processor system 906 for and I/O interface 908 for input and output of data, such as data communicated between hemodynamic monitoring system 900 with PPG 902, pump system 904, and a user interface. Hemodynamic monitoring system 900 can utilize a number of applications stored within a memory system 910 to be executed by processor system 906. Applications that can be stored within a memory system 910 including real-time hemodynamic data applications 912, calibration applications 914, and pressure regulation applications 916 for operating the hemodynamic monitoring system.

While a specific hemodynamic monitoring system configuration is described above with reference to FIG. 7, it should be readily appreciated that various hemodynamic monitoring systems and/or other medical monitoring utilized in the provision of hemodynamic monitoring can be implemented in any of a variety of configurations. Accordingly, the various systems and methods described herein should be understood as not to be limited to any specific hemodynamic monitoring system, but instead can be implemented using any variety of hemodynamic monitoring or medical monitoring systems capable of measuring arterial blood pressure within a digit.

In various examples, the portable blood pressure monitoring system 400 can measure continuous blood pressure using a volume clamp method for 95% of a given patient population. In various examples, an automatic mechanism may provide a consistent preload with the volume clamp method. In various examples, a fixed LED and photodiode positions provide guaranteed photoplethysmogram signal alignment. In various examples, the portable blood pressure monitoring system 400 improves the usability of the finger cuff patient interface for healthcare professionals by tightening the cuff for them.

In various examples, the portable blood pressure monitoring system 400 provides quick and easy setup of the system in 30 seconds or less.

While the above description contains many specific implementations of the invention, these should not be construed as limitations on the scope of the disclosure, but rather as an example of one implementation thereof. It is therefore to be understood that the present disclosure may be practiced in ways other than specifically described, without departing from the scope and spirit of the present disclosure. Thus, implementations of the present disclosure should be considered in all respects as illustrative and not restrictive. Accordingly, the scope of the disclosure should be determined not by the implementations illustrated, but by the appended claims and their equivalents.

What is claimed is:

1. A blood pressure monitor component for monitoring a patient's blood pressure, comprising:
a cuff defining a cavity for receiving an appendage;
a bladder at least partially surrounding the cavity;
a fixed end of the cuff connected with a base and a free end of the cuff movable with respect to the fixed end to adjust a diameter of the cavity; and
a force sensor configured to sense tension applied by the cuff.

2. The blood pressure monitor component of claim 1, further comprising:
a rack which includes a series of apertures along the free end of the cuff;
a pinion capable of engaging with the series of apertures of the rack; and
a computer comprising:
a processor; and
memory with instructions configured to operate the processor to:
receive force data from the force sensor;
determine an amount of tension being applied by the cuff by the force data; and
operate the pinion based on the amount of tension being applied by the cuff.

3. The blood pressure monitor component of claim 2, further comprising a light source and a light sensor which are integrated within the cuff, wherein the light source is configured to output light which is received by the light sensor, wherein the amount of light which is received by the light sensor corresponds to the blood pressure.

4. The blood pressure monitor component of claim 3, wherein the instructions are further configured to operate the processor to perform a blood pressure measurement after operating the pinion.

5. The blood pressure monitor component of claim 2, further comprising a motor attached to a gear by a shaft, wherein the gear drives the pinion.

6. The blood pressure monitor component of claim 5, wherein operating the pinion based on the amount of tension being applied by the cuff comprises upon a determination that the amount of tension being applied by the cuff is too high, operating the motor which drives the pinion to adjust the cuff to be loosened.

7. The blood pressure monitor component of claim 5, wherein operating the pinion based on the amount of tension being applied by the cuff comprises upon a determination that the amount of tension being applied by the cuff is too low, operating the motor which drives the pinion to adjust the cuff to be tightened.

8. The blood pressure monitor component of claim 5, wherein operating the pinion based on the amount of tension being applied by the cuff comprises upon a determination that the amount of tension being applied by the cuff is appropriate, operating the motor which drives the pinion to maintain the tension.

9. The blood pressure monitor component of claim 8, wherein operating the motor which drives the pinion to maintain the tension comprises providing no signal to the motor.

10. The blood pressure monitor component of claim 5, wherein the pinion includes teeth which corresponds to the series of apertures of the rack to move the rack and decrease or increase the size of the cuff.

11. The blood pressure monitor component of claim 5, wherein the gear includes teeth which interlock with the teeth of the pinion.

12. The blood pressure monitor component of claim 2, wherein the pinion is capable of rotation, wherein rotation of the pinion results in linear movement of the cuff such that cuff is tightened or loosened.

13. A blood pressure monitor system for monitoring a patient's blood pressure, comprising:
a blood pressure monitor component comprising:
a cuff connected to a rack which includes a series of apertures along at least one end of the cuff, wherein the cuff comprises a bladder;
a pinion capable of engaging with the series of apertures of the rack; and
a force sensor configured to sense tension applied by the cuff; and
a pump system in operable connection with the bladder configured to provide pressure to the bladder.

14. The blood pressure monitor system of claim 13, further comprising a computer comprising:
a processor; and
memory with instructions configured to operate the processor to:
receive force data from the force sensor;
determine an amount of tension being applied by the cuff by the force data; and
operate the pinion based on the amount of tension being applied by the cuff.

15. The blood pressure monitor system of claim 14, wherein operating the pinion based on the amount of tension being applied by the cuff comprises upon a determination that the amount of tension being applied by the cuff is too high, operating the motor which drives the pinion to adjust the cuff to be loosened.

16. The blood pressure monitor system of claim 14, wherein operating the pinion based on the amount of tension being applied by the cuff comprises upon a determination that the amount of tension being applied by the cuff is too low, operating the motor which drives the pinion to adjust the cuff to be tightened.

17. The blood pressure monitor system of claim 14, wherein the instructions are further configured to operate the processor to operate the pump system to provide an amount of pressure to the bladder.

18. The blood pressure monitor system of claim 13, further comprising a light source and a light sensor which are integrated within the cuff, wherein the light source is configured to output light which is received by the light sensor, wherein the amount of light which is received by the light sensor corresponds to the blood pressure.

19. The blood pressure monitor system of claim 18, wherein the instructions are further configured to operate the processor to perform a blood pressure measurement after operating the pinion.

20. The blood pressure monitor system of claim 13, further comprising a motor attached to a gear by a shaft, wherein the gear drives the pinion.
